(19) United States
(12) Patent Application Publication
(10) Pub. No.: US 2018/0021230 A1
(43) Pub. Date: Jan. 25, 2018

(54) LEAD, CHALK OR COLOR TABLET FOR WRITING, DRAWING AND/OR COSMETICS PURPOSES, PENCIL CONTAINING A LEAD, AND METHOD FOR PRODUCING A LEAD, CHALK OR COLOR TABLET

(71) Applicant: FABER-CASTELL AG, STEIN (DE)

(72) Inventors: HARALD VON GODIN, OBERASBACH (DE); REINER APPEL, OBERASBACH (DE); GERHARD LUGERT, NUERNBERG (DE)

(21) Appl. No.: 15/591,143

(22) Filed: May 10, 2017

(30) Foreign Application Priority Data
Jul. 20, 2016 (EP) 16180428.1

(51) Int. Cl. A61K 8/34; A61K 8/46; A61K 8/25; C09D 13/00; A61K 8/92; A61K 8/96; A61K 8/60; A61Q 1/02; A61K 8/73; A61K 8/29

(52) U.S. Cl. CPC A61K 8/345; A61K 8/731; A61K 8/466; A61K 8/25; A61K 8/29; A61K 8/732; A61K 8/463; A61K 8/92; A61K 8/965; A61K 8/60; A61Q 1/02; C09D 13/00; A61K 2800/43; A61K 2800/872

(57) ABSTRACT

A lead or chalk or color tablet for writing, drawing and/or cosmetics purposes, contains at least one sugar and/or sugar alcohol and/or pentaerythritol, at least one cellulose derivative and/or starch derivative and at least one surfactant. The at least one surfactant is selected from the group of sulfuric acid fatty acid ester salts and/or fatty acid sulfoalkyl esters and/or ethoxylated forms of alkali metal fatty acid sulfates. A pencil having such a lead and to a method for producing a lead or chalk or color tablet are also provided.

### CROSS-REFERENCE TO RELATED APPLICATION

This application claims the priority, under 35 U.S.C. §119, of European Patent Application EP 16 180 428.1, filed Jul. 20, 2016; the prior application is herewith incorporated by reference in its entirety.

### BACKGROUND OF THE INVENTION

### Field of the Invention

The invention relates to a lead, chalk or color tablet for writing, drawing and/or cosmetics purposes and also to a pencil containing such a lead. Furthermore, the invention relates to a method for producing a lead, chalk or color tablet.

European Patent EP 1 552 958 B1, corresponding to U.S. Patent Application Publication US 2005/0053633 A1, discloses leads produced by thermoplastic processing of a lead starting composition containing sugars and/or sugar alcohols and also cellulose derivatives as binders. Such leads or lead starting compositions are virtually anhydrous and can therefore be stored for multiple weeks or months. Furthermore, there is hardly any cause for concern with regard to microbiological impairment due to the absent water content. Leads having such a binder system exhibit a good breaking strength and are additionally distinguished by a very uniform, homogeneous application on a writing or drawing base. However, the applications on paper and drawing cardboard of the thermoplastic leads described in European Patent EP 1 552 958 B1, corresponding to U.S. Patent Application Publication US 2005/0053633 A1, show only a satisfactory to good aquarellability. Thus, the color application cannot be subsequently optimally finely or broadly spread and the luminance therefore cannot fully develop. Also, it has so far not been possible to consistently guarantee a very good lightfastness of the color applications on paper or drawing cardboard. However, precisely excellent aquarellabilities and very good to excellent lightfastnesses are desired for colored pencils used by artists.

### SUMMARY OF THE INVENTION

It is accordingly an object of the invention to provide a lead, chalk or color tablet for writing, drawing and/or cosmetics purposes which overcomes the hereinafore-mentioned disadvantages of the heretofore-known devices of this general type and which is improved especially with respect to aquarellability and lightfastness. Furthermore, it is an object of the invention to specify a method for producing a lead, chalk or color tablet and also a pencil containing a lead.

With the foregoing and other objects in view there is provided, in accordance with the invention, a lead or chalk or color tablet for writing, drawing and/or cosmetics purposes, comprising at least one sugar and/or sugar alcohol and/or pentaerythritol, at least one cellulose derivative and/or starch derivative and at least one surfactant, the at least one surfactant being selected from the group of sulfuric acid fatty acid ester salts (=fatty alcohol sulfates) and/or fatty acid sulfoalkyl esters and/or ethoxylated forms of alkali metal fatty acid sulfates.

Hereinafter, reference is simplistically predominantly made to a lead, but the explanations similarly also apply to a chalk or a color tablet. The lead or chalk or color tablet is especially an aquarellable lead or chalk or color tablet or a watercolor.

The lead therefore contains, as binder, a binder system containing substantially sugar and/or sugar alcohol and/or pentaerythritol and a cellulose derivative and/or starch derivative. The lead base composition or the lead starting composition, i.e., the at least one sugar and/or sugar alcohol and/or pentaerythritol, the at least one cellulose derivative and/or starch derivative and the at least one surfactant and also optionally pigments and/or fillers and/or further additives, is thermoplastically processed at an elevated temperature bringing about a softening of the sugar and/or sugar alcohol and/or pentaerythritol in order to produce the lead. In this connection, water added during the production of the lead in order to promote mixing of the starting substances is, if necessary, reduced again to a fraction of not more than 2% by weight. The lead itself or the lead base composition is anhydrous and contains at most 2% by weight of water.

The lead is therefore produced from a lead base composition or lead starting composition which contains the stated components or starting substances and which is thermoplastically processed and extruded in order to melt and to bind the starting substances. The lead strands obtained therefrom are then optionally cut to length. Cooling of the lead composition gives the finished lead. A complicated removal of water or solvents is not necessary.

To date, the use or the addition of surfactants in conventional leads or lead compositions containing sugars and/or sugar alcohols and/or pentaerythritol and also at least one cellulose derivative and/or starch derivative and also optionally pigments and/or fillers has been avoided, since it is known that surfactants, for example silicone surfactants or fluorosurfactants, in combination with such a binder system lead to a deterioration of the mechanical stability of the lead or colored lead without bringing along other advantages.

However, it has been possible to establish that, surprisingly, such an effect does not occur in surfactants from the group of the sulfuric acid fatty acid ester salts and/or fatty acid sulfoalkyl esters and/or ethoxylated forms of alkali metal fatty acid sulfates. On the contrary, it was possible to achieve distinct improvements in the aquarellability of the leads produced therefrom without a noticeable impairment in mechanical stability occurring. In addition, very good to excellent lightfastnesses of the applications consistently occurred. Moreover, in most cases, improved processing properties in the extrusion of the lead base composition and the leads developed therefrom were even achieved. This can probably be attributed to the fact that precisely the selected surfactants are suitable for promoting a combining or mixing of the components of the binder, specifically the sugar and/or sugar alcohol and the cellulose derivative and/or starch derivative.

The cations of the stated surfactants that are used are monovalent metal cations such as, for example, potassium, sodium, lithium, but also ammonium. Preferably, the surfactants or the sulfuric acid fatty acid ester salts that are used are sodium lauryl sulfate, potassium lauryl sulfate, ammonium lauryl sulfate, salts such as sodium cocoyl isethionate or sodium myreth sulfate. One ethoxylated form of an alkali metal fatty acid sulfate that is preferably used is sodium dodecylpoly(oxyethylene) sulfate.

The sugar which is used is preferably sucrose and/or xylose. However, in order to achieve a reliable combining of the sugar with the cellulose derivative and/or starch derivative, an elevated pressure in the extruder is often necessary during the thermoplastic processing or the melting of the two components, since numerous sugars such as, for example, glucose, fructose, sucrose, mannose and also various cellulose derivatives and/or starch derivatives exhibit signs of decomposition at a processing temperature between 100 and 220° C. This can be avoided through the use of sugar alcohols, more particularly mannitol, sorbitol, xylitol, adonitol, arabitol, dulcitol or threitol, which can at first melt in a water-clear manner above 100° C. without application of elevated pressure and, surprisingly, incorporate celluloses or starches or their derivatives without any problems. Especially the sugar alcohol mannitol shows a very good processability and has therefore been found to be particularly advantageous. The use of a single sugar or sugar alcohol is just as conceivable as the use of two or more sugars or sugar alcohols in combination.

Sodium carboxymethylcellulose (NaCMC) and/or carboxymethyl starch (CMS) and/or sodium carboxymethyl starch (NaCMS) have been found to be particularly useful as cellulose derivatives and/or starch derivatives. A temperature of about from 80 to 150° C. is sufficient for mixing or combining the two components, as the sugar and/or sugar alcohol and the cellulose derivative and/or starch derivative, which jointly act as binder. Especially due to addition of water during the production of the lead, the cellulose derivatives and/or starch derivatives swell and thereby bind further starting substances.

In a preferred embodiment, the lead or chalk or color tablet contains from 1 to 30% by weight, more particularly from 1 to 25% by weight, of surfactant. As already described, it was surprising that the use of a surfactant from the group of the sulfuric acid fatty acid ester salts and/or fatty acid sulfoalkyl esters and/or ethoxylated forms of alkali metal fatty acid sulfates in the present binder system distinctly improves the aquarellability and lightfastness of the leads, but nevertheless does not lead to any appreciable deterioration in the mechanical stability of the leads. This interaction or this effect is advantageously ensured by the fraction of the surfactant in the lead or in the lead base composition not exceeding 30% by weight, more particularly 25% by weight.

Preferably, the lead or chalk or color tablet contains from 4 to 40% by weight, more particularly from 4 to 30% by weight, of sugar and/or sugar alcohol and/or pentaerythritol. Limiting the fraction of sugar to a maximum of 40% by weight, more particularly to a maximum of 30% by weight, makes it possible to reliably avoid a deterioration in the processability of the lead base composition and a smaller breaking strength of the lead.

Furthermore, in a preferred embodiment, the lead contains from 0.5 to 25% by weight, more particularly from 0.5 to 18% by weight, of cellulose derivative and/or starch derivative. Limiting the fraction of cellulose derivative and/or starch derivative to a maximum of 25% by weight, more particularly to a maximum of 18% by weight, makes certain that a good processability, more particularly a good mixing of the starting substances or, more particularly, of the two binders, is ensured.

Adding one or more fillers can influence the property profile of the lead. In this connection, the addition of a filler at a fraction of from 30 to 80% by weight, more particularly at a fraction of from 40 to 70% by weight, to the lead base composition has been found to be advantageous. Suitable fillers are, in particular, talc, kaolin, pumice powder, calcium carbonate, barium sulfate, mica and/or titanium dioxide.

If a surfactant present in the lead already has waxy properties, it may be possible to dispense with the addition of fatty or waxy substances. For example, sodium cocoyl isethionate exhibits such waxy properties in the use as surfactant in combination with the further constituents of the lead or the lead base composition. However, in order to be able to further vary the property profile of the lead, for instance its application behavior, the lead base composition contains, in a preferred variant, at least one fatty or waxy substance, especially at a fraction of from 0.1 to 30% by weight. In this connection, the fatty or waxy substance is preferably at least one fatty acid and/or one fatty acid derivative and/or one wax. The use of fatty or waxy substances having both lipophilic and hydrophilic properties makes it possible to produce water-soluble or water-swellable leads. This therefore yields leads corresponding in terms of their properties to the known so-called “post-wax leads,” which are immersed in melted wax or oil after their production. That additional working step is omitted in this case, simplifying the production of the leads, in particular shortening the time required for the production of the lead. Fatty acids such as stearic acid, paraffin waxes, Japan wax substitute or ethoxy (80) cetostearyl alcohol, other fatty acid derivatives or hydrocarbon wax are, for example, used as such fatty or waxy substances.

In order to obtain colored leads or chalks or color tablets, the lead base composition contains at least one colorant, more particularly an inorganic and/or organic pigment, especially at a fraction of from 0.5 to 20% by weight.

In a further preferred variant, the lead contains at least one inorganic and/or organic pigment and at least one water-soluble dye in combination. Although the addition of water-soluble dyes reduces the excellent lightfastness, it allows the production of so-called copying pencil leads or the use of the lead in a copying pencil.

If necessary, the lead can contain further additives, such as, for example, lubricants or emulsifiers, which are added to the lead base composition during the production of the lead in order to further vary the property profile of the lead.

With the objects of the invention in view, there is also provided a pencil, comprising a lead having the above-described features. In this connection, the pencil has in particular a sheathing which is composed of wood or composed of a wood substitute material and which encases the lead. The wood substitute material which is used instead of natural wood is, for example, a so-called wood-plastic compound, and so it is, for example, possible for the pencil to be produced in a simple manner by coextrusion of the lead and of the sheathing.

With the objects of the invention in view, there is concomitantly provided a method for producing a lead or a chalk or a color tablet in which the starting substances required for producing the lead or the substances present in the lead base composition, i.e., the at least one sugar and/or sugar alcohol and/or pentaerythritol, the at least one cellulose derivative and/or starch derivative and the at least one surfactant, are mixed, and thermoplastically processed or mixed and combined with one another at an elevated temperature bringing about a softening of the sugar and/or of the sugar alcohol and/or of the pentaerythritol. Optionally, further substances, such as, for example, fillers or a fatty or waxy substance, are added and jointly thermoplastically processed with the above-mentioned starting substances. The lead base composition obtained therefrom is extruded to give leads or chalk strands and/or the lead base composition obtained therefrom is shaped to give color tablets. The lead base composition containing at least one surfactant selected from the group of the sulfuric acid fatty acid ester salts and/or fatty acid sulfoalkyl esters and/or ethoxylated forms of alkali metal fatty acid sulfates can be thermoplastically processed better than conventional leads containing no surfactant. The surfactant leads to a better mixability and more intimate combining of the starting substances.

Preferably, water which is added to the starting substances is jointly thermoplastically processed with the at least one sugar and/or sugar alcohol and/or pentaerythritol, the at least one cellulose derivative and/or starch derivative and the at least one surfactant, with the fraction of the water being reduced to at most 2% by weight during the thermoplastic processing, for instance in compounding in an extruder. In other words: the water which is present and which is added to the starting substances, for example at a fraction of from 5 to 30% by weight, in order to promote the mixing or compounding process of the sugar and/or sugar alcohol and/or pentaerythritol, of the at least one cellulose derivative and/or starch derivative and of the at least one surfactant and also optionally further substances is evaporated up to a residual content of less than or equal to 2% by weight, and so it is then possible to melt the sugar or sugar alcohol or pentaerythritol, with this melting naturally not being achievable in aqueous solution. The extruded or shaped lead base composition has a low moisture content as a result of the reduction of the water content, and therefore cooling of the lead base composition gives the finished lead. A complicated removal of water or other solvents is omitted.

Other features which are considered as characteristic for the invention are set forth in the appended claims.

Although the invention is described herein as embodied in a lead, a chalk or a color tablet for writing, drawing and/or cosmetics purposes, a pencil containing a lead and a method for producing a lead, chalk or color tablet, it is nevertheless not intended to be limited to the details provided, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying examples.

### DESCRIPTION OF THE INVENTION

Four exemplary recipes for a lead are listed below. The starting substances or raw materials are first mixed at a temperature of from 80 to 150° C. in a high-speed mixer or in an extruder. In order to quicken the mixing or homogenization process, a water fraction of from 15 to 20% by weight is added and is evaporated up to a residual moisture of not more than 2% by weight during the mixing operation in the high-speed mixer or extruder. The lead base composition which is obtained is extruded at about from 140 to 180° C. in a screw extruder and then cut to length to give leads. The leads or chalks thus produced have a diameter of between 2.6 and 6.0 mm.

### Example 1

### Aquarellable Green Colored Lead or Aquarellable Green Chalk:

| | |
|---|---|
| Mannitol | 6.0% by weight |
| NaCMC¹⁾ | 4.0% by weight |
| Sodium cocoyl isethionate | 20.0% by weight |
| (CAS 61789-32-0) | |
| Kaolin | 56.4% by weight |
| Titanium dioxide | 5.0% by weight |
| Pigment Yellow 3 | 1.8% by weight |
| Pigment Yellow 1 | 0.9% by weight |
| Pigment Blue 15 | 1.35% by weight |
| Pigment Green 7 | 4.55% by weight |

A lead or chalk or a pencil containing a lead having a composition in line with Example 1 has a very good processability or extrudability. This can be attributed in particular to the use of mannitol as sugar alcohol and to the high fraction of 20% by weight of the surfactant used (sodium cocoyl isethionate). Furthermore, the lead exhibits an excellent aquarellability and lightfastness.

### Example 2

### Aquarellable Blue Colored Lead or Aquarellable Blue Chalk:

| | |
|---|---|
| Sorbitol | 6.7% by weight |
| Carboxymethyl starch²⁾ | 4.5% by weight |
| Sodium myreth sulfate | 10.0% by weight |
| (CAS 25446-80-4) | |
| Hydrocarbon wax | 6.8% by weight |
| Pumice powder | 63.2% by weight |
| Titanium dioxide | 1.0% by weight |
| Pigment Blue 15 | 7.2% by weight |
| Pigment Red 146 | 0.6% by weight |

In the case of a lead or chalk or a pencil containing a lead having a composition in line with Example 2, the fraction of surfactant, sodium myreth sulfate in this case, was reduced as compared to Example 1. Nevertheless, a sufficiently good processability and extrudability with comparable aquarellability and lightfastness is also apparent in the production of the lead.

### Example 3

### Aquarellable Red Colored Lead or Aquarellable Red Chalk:

| | |
|---|---|
| Xylitol | 7.0% by weight |
| Carboxymethyl starch²⁾ | 5.0% by weight |
| Sodium lauryl sulfoacetate | 8.0% by weight |
| (CAS 1847-58-1) | |
| Sorbitan monostearate | 8.0% by weight |
| Talc | 62.6% by weight |
| Titanium dioxide | 1.0% by weight |
| Pigment Red 254 | 7.0% by weight |
| Pigment Yellow 3 | 1.4% by weight |

In the case of a lead or chalk or a pencil containing a lead having a composition in line with Example 3, the surfactant used was sodium lauryl sulfoacetate and its fraction was further reduced as compared to Examples 1 and 2, specifically to 8% by weight. However, aquarellability and lightfastness and also processability are also still satisfactory in this case.

### Example 4

### Aquarellable Orange Colored Lead or Aquarellable Orange Chalk:

| | |
|---|---|
| Mannitol | 17.4% by weight |
| Carboxymethyl starch²⁾ | 2.3% by weight |
| Sodium lauryl sulfate | 15.0% by weight |
| (CAS 151-21-3) | |
| Hydrocarbon wax | 4.3% by weight |
| Kaolin | 50.6% by weight |
| Titanium dioxide | 6.0% by weight |
| Pigment Yellow 74 | 3.6% by weight |
| Pigment Orange 34 | 0.8% by weight |

In the case of a lead or chalk or a pencil containing a lead having a composition in line with Example 4, the surfactant used was sodium lauryl sulfate. Such a lead or chalk exhibits in particular a good aquarellability.

Product Names/Manufacturers:

### 1) Dow Wolff Cellulosics GmbH, 29699 Bornlitz, Germany

### 2) Südstärke GmbH, 86529 Schrobenhausen, Germany

1. A lead or chalk or color tablet for at least one of writing, drawing or cosmetics purposes, comprising:
at least one of at least one sugar or sugar alcohol or pentaerythritol;
at least one of at least one cellulose derivative or starch derivative; and
at least one surfactant selected from the group consisting of sulfuric acid fatty acid ester salts, fatty acid sulfoalkyl esters and ethoxylated forms of alkali metal fatty acid sulfates.

2. The lead or chalk or color tablet according to claim 1, which further comprises providing at least one of sodium lauryl sulfate, potassium lauryl sulfate, ammonium lauryl sulfate, sodium cocoyl isethionate, sodium myreth sulfate or sodium dodecylpoly(oxyethylene) sulfate as the surfactant.

3. The lead or chalk or color tablet according to claim 1, which further comprises providing at least one of sucrose or xylose as the sugar.

4. The lead or chalk or color tablet according to claim 1, which further comprises providing mannitol, sorbitol, xylitol, adonitol, arabitol, dulcitol or threitol as the sugar alcohol.

5. The lead or chalk or color tablet according to claim 1, which further comprises providing at least one of sodium carboxymethylcellulose or carboxymethyl starch or sodium carboxymethyl starch as at least one of the cellulose derivative or starch derivative.

6. The lead or chalk or color tablet according to claim 1, which further comprises providing from 1 to 30% by weight of the surfactant.

7. The lead or chalk or color tablet according to claim 1, which further comprises providing from 1 to 25% by weight of the surfactant.

8. The lead or chalk or color tablet according to claim 1, which further comprises providing from 4 to 40% by weight of at least one of the sugar or sugar alcohol or pentaerythritol.

9. The lead or chalk or color tablet according to claim 1, which further comprises providing from 4 to 30% by weight of at least one of the sugar or sugar alcohol or pentaerythritol.

10. The lead or chalk or color tablet according to claim 1, which further comprises providing from 0.5 to 25% by weight of at least one of the cellulose derivative or starch derivative.

11. The lead or chalk or color tablet according to claim 1, which further comprises providing from 0.5 to 18% by weight of at least one of the cellulose derivative or starch derivative.

12. The lead or chalk or color tablet according to claim 1, which further comprises providing at least one filler at a fraction of from 30 to 80% by weight.

13. The lead or chalk or color tablet according to claim 1, which further comprises providing at least one filler at a fraction of from 40 to 70% by weight.

14. The lead or chalk or color tablet according to claim 12, which further comprises selecting the filler from at least one of the group consisting of talc, kaolin, pumice powder, calcium carbonate, barium sulfate, mica and titanium dioxide.

15. The lead or chalk or color tablet according to claim 13, which further comprises selecting the filler from at least one of the group consisting of talc, kaolin, pumice powder, calcium carbonate, barium sulfate, mica and titanium dioxide.

16. The lead or chalk or color tablet according to claim 1, which further comprises providing at least one fatty or waxy substance.

17. The lead or chalk or color tablet according to claim 16, which further comprises providing the at least one fatty or waxy substance at a fraction of from 0.1 to 30% by weight.

18. The lead or chalk or color tablet according to claim 16, wherein the at least one fatty or waxy substance contains at least one of a fatty acid or a fatty acid derivative or a wax.

19. The lead or chalk or color tablet according to claim 17, wherein the at least one fatty or waxy substance contains at least one of a fatty acid or a fatty acid derivative or a wax.

20. The lead or chalk or color tablet according to claim 1, which further comprises at least one colorant.

21. The lead or chalk or color tablet according to claim 20, wherein the at least one colorant is at least one of an inorganic or organic pigment.

22. The lead or chalk or color tablet according to claim 20, which further comprises providing the at least one colorant at a fraction of from 0.5 to 20% by weight.

23. The lead or chalk or color tablet according to claim 13, which further comprises providing at least one of at least one inorganic or organic pigment and at least one water-soluble dye.

24. A pencil, comprising a lead according to claim 1.

25. The pencil according to claim 24, which further comprises a sheathing composed of wood or a wood substitute material.

26. A method for producing a lead or a chalk or a color tablet for at least one of writing, drawing or cosmetics purposes, the method comprising the following steps:
providing at least one of at least one sugar or sugar alcohol or pentaerythritol;
providing at least one of at least one cellulose derivative or starch derivative;
providing at least one surfactant selected from the group consisting of sulfuric acid fatty acid ester salts, fatty acid sulfoalkyl esters and ethoxylated forms of alkali metal fatty acid sulfates;
thermoplastically processing the at least one of at least one sugar or sugar alcohol or pentaerythritol, the at least one of at least one cellulose derivative or starch derivative and the at least one surfactant at an elevated temperature bringing about a softening of at least one of the sugar or sugar alcohol or of the pentaerythritol and obtaining a lead base composition; and
extruding the lead base composition to give at least one of leads or chalk strands or shaping the lead base composition to give color tablets.

27. The method according to claim 26, which further comprises:
adding water being jointly mixed with the at least one of at least one sugar or sugar alcohol or pentaerythritol, the at least one of at least one cellulose derivative or starch derivative and the at least one surfactant; and
reducing a fraction of the water to at most 2% by weight during the thermoplastic processing.
